(19) United States
(12) Patent Application Publication
(10) Pub. No.: US 2017/0065201 A1
(43) Pub. Date: Mar. 9, 2017

(54) HUMAN BODY IMPEDANCE MEASUREMENT DEVICE

(71) Applicant: MSP CO., LTD, Incheon (KR)

(72) Inventor: Moon Seo Park, Incheon (KR)

(73) Assignee: MSP CO., LTD, Incheon (KR)

(21) Appl. No.: 15/121,876

(22) PCT Filed: Feb. 27, 2015
(86) PCT No.: PCT/KR2015/001919

§ 371 Date: Oct. 19, 2016

(30) Foreign Application Priority Data
Feb. 28, 2014 (KR) 10-2014-0024371
Feb. 28, 2014 (KR) 10-2014-0024372

(51) Int. Cl. A61B 5/053

(52) U.S. Cl. CPC A61B 5/0536

(57) ABSTRACT

Disclosed is a device for measuring impedance within a human body, including: a spiral base plate; a plurality of electrodes arranged along the spiral base plate; and a plurality of first and second power lines connected to the plurality of electrodes. According to the present invention, the spiral base plate is used, so that it is possible to easily apply the device in accordance with a curve of a human body.

### TECHNICAL FIELD

The present invention relates to a device for a human body impedance measurement, and more particularly, to a device for measuring human body impedance data by being easily applied to a human body, and a device for 3D imaging by using the data.

### BACKGROUND ART

Recently, Electrical Impedance Tomography (EIT) is getting the spotlight, and hardware of the EIT is relatively cheap when a system is implemented, and the EIT has a nondestructive characteristic for a measurement target object. In the EIT, spatial resolution of a restored image is still low compared to X-ray and Magnetic Resonance Imaging (MRI) tomography, but temporal resolution is high and safety to a human body is guaranteed, so that the EIT is used as auxiliary equipment in a medical engineering field.

The EIT is a method of measuring resistance of a bodily tissue after making a current of several millivolt amperes of 10 to 100 KHz flow into a human body, and in order to recognize an electrical characteristic of a body section, several electrodes are attached to body parts, a current sequentially flows, resistance is measured, and then the corresponding resistance is imaged.

However, in the EIT, in order to make a current flow to a body, an electrode needs to be in direct contact with the body so that it is difficult in that a substrate needs to be manufactured in consideration of a shape of a part of the body, to which the EIT is desired to be applied.

### DISCLOSURE

### Technical Problem

The present invention has been made in an effort to provide a device for a human body impedance measurement, which is easily applied to a curve part of the human body.

Further, the present invention provides a device for a human body impedance measurement having a structure, in which electrodes may be easily arranged to be perpendicular to one another.

Further, the present invention provides a device for a human body impedance measurement, which is capable of obtaining data related to a 3D shape of a human body by efficiently detecting stress according to a curve of the human body through the small number of sensors.

### Technical Solution

In an aspect, a device for measuring impedance within a human body according to the present invention includes: a spiral base plate; a plurality of electrodes arranged along the spiral base plate; and a plurality of first and second power lines connected to the plurality of electrodes.

A stress sensor, which detects a degree of bending of the spiral base plate, is provided at least a part of spaces between the electrodes arranged along the spiral base plate.

Further, the device may further include a shape calculating unit configured to calculate a 3D shape of the human body, to which the base plate is applied, from data about the degree of bending for each part of the base plate transmitted from the stress sensor.

The plurality of electrodes may be arranged from a center of the base plate in a radial direction.

The base plate may include: a plurality of circular band bases, which is formed to have a gradually increased radius and is spaced apart from one another by a predetermined interval, and has a gap portion having a radially incised shape at a part of the circular band base; and a base bridge configured to connect an end portion of any one of the plurality of circular band bases and an end portion of an adjacent outer circular band base.

An average interval of the gap portion may be 5 mm to 20 mm.

An interval of the gap portion may be formed so as to correspond to a distance between the electrodes.

A stress sensor, which detects a degree of bending of the base bridge, may be provided on the base bridge.

The distance between the electrodes may be 5 mm to 20 mm.

The first power line and the second power line may be an input electrode and a receiving electrode, respectively.

In another aspect, a device for measuring impedance within a human body according to the present invention includes: a flexible base plate; a plurality of electrodes arranged in a first direction and a second direction orthogonal to the first direction on the flexible base plate; a plurality of stress sensors arranged on the flexible base plate and configured to detect a degree of bending of the flexible base plate; and a plurality of first and second power lines connected to the plurality of electrodes.

The flexible base plate may be formed with branch-leaf portions radially extended from a center portion, and the electrode and the stress sensor may be arranged on the branch-leaf portion.

The flexible base plate may be spirally formed, and the electrode and the stress sensor may be arranged along the spiral flexible base plate.

The base plate may include: a plurality of circular band bases, which is formed to have a gradually increased radius and is spaced apart from one another by a predetermined interval, and has a gap portion having a radially incised shape at a part of the circular band base; and a base bridge configured to connect an end portion of any one of the plurality of circular band bases and an end portion of an adjacent outer circular band base.

The stress sensor may be provided on the base bridge and detect a degree of bending of the base bridge.

Further, the device may further include a shape calculating unit configured to calculate a 3D shape of the human body, to which the base plate is applied, from data about the degree of bending for each part of the base plate transmitted from the stress sensor.

The plurality of electrodes may be arranged from a center of the base plate in a radial direction.

The distance between the electrodes may be 5 mm to 20 mm.

The first power line and the second power line may be an input electrode and a receiving electrode, respectively.

### Advantageous Effect

According to the present invention, the spiral base plate is used, so that it is possible to easily apply the device in accordance with a curve of a human body.

Further, according to the present invention, a structure, in which the bases are formed in the shape of the plurality of circular bands, and the circular band bases are connected through the base bridges, is introduced, so that it is possible to easily arrange the wires of the electrodes so as to be orthogonal to each other.

Further, according to the present invention, it is possible to detect stress applied to the base plate according to a curve of a human body, thereby recognizing a 3D shape of the human body that is a measurement target.

Further, according to the present invention, it is possible to efficiently detect stress according to a curve of a human body by using the small number of sensors by concentrating the stress sensor to the base bridge, in which the stress is concentrated, thereby obtaining data related to a 3D shape of the human body.

### DESCRIPTION OF DRAWINGS

FIG. 1 is a top plan view illustrating a human body impedance measurement device including a stress sensor according to an exemplary embodiment.

FIG. 2 is a cross-sectional view illustrating electrodes provided in the human body impedance measurement device according to the exemplary embodiment.

FIG. 3 is a cross-sectional view illustrating an operation state of the electrodes provided in the human body impedance measurement device according to the exemplary embodiment.

FIGS. 4 to 7 are schematic diagrams for describing a process of imaging impedance inside a human body by using an impedance value measured by Electrical Impedance Tomography (EIT).

FIG. 8 is a perspective view illustrating a spiral human body impedance measurement device according to an exemplary embodiment of the present invention.

FIG. 9 is a bottom perspective view illustrating the spiral human body impedance measurement device according to the exemplary embodiment.

FIGS. 10 and 11 are schematic diagrams illustrating an application of the spiral human body impedance measurement device according to the exemplary embodiment to a human body.

FIG. 12 is a top plan view illustrating a spiral human body impedance measurement device according to another exemplary embodiment.

### BEST MODE

A human body impedance measurement device according to the present invention includes: a spiral base plate; a plurality of electrodes arranged along the spiral base plate; and a plurality of first and second power lines connected to the plurality of electrodes.

### MODE FOR CARRYING OUT THE INVENTION

Hereinafter, exemplary embodiments of the present invention will be described with reference to the accompanying drawings. Unless there is a special definition or mention, terms indicating a direction used in the present description are based on a state illustrated in the drawing. Further, the same reference numeral designates the same member throughout each exemplary embodiment. In the meantime, for convenience of the description, a thickness or a size of each constituent element illustrated in the drawings may be exaggerated, and it does not mean that the constituent element needs to be actually configured with a corresponding size or a ratio between the elements.

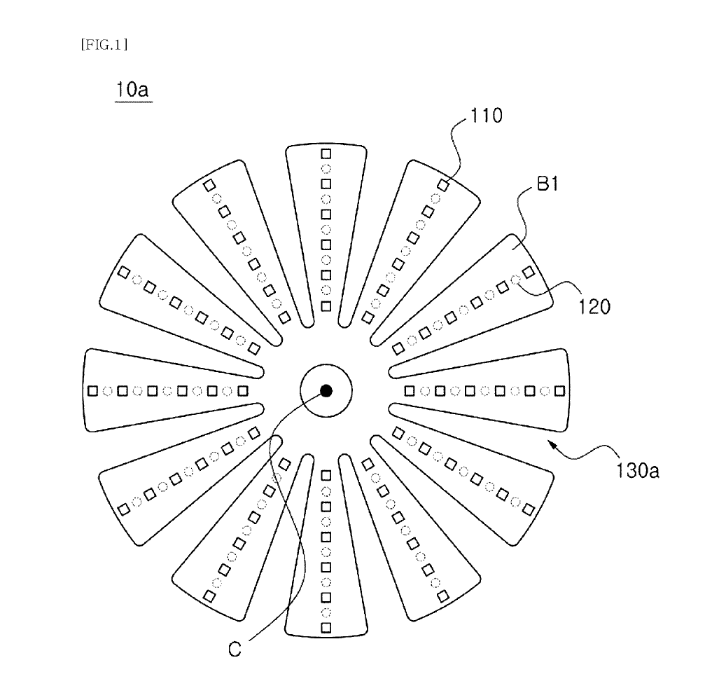

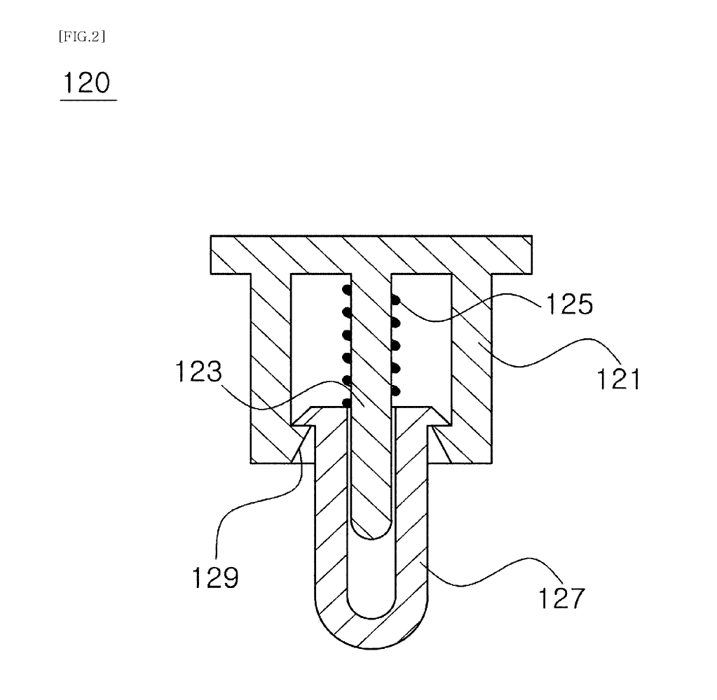

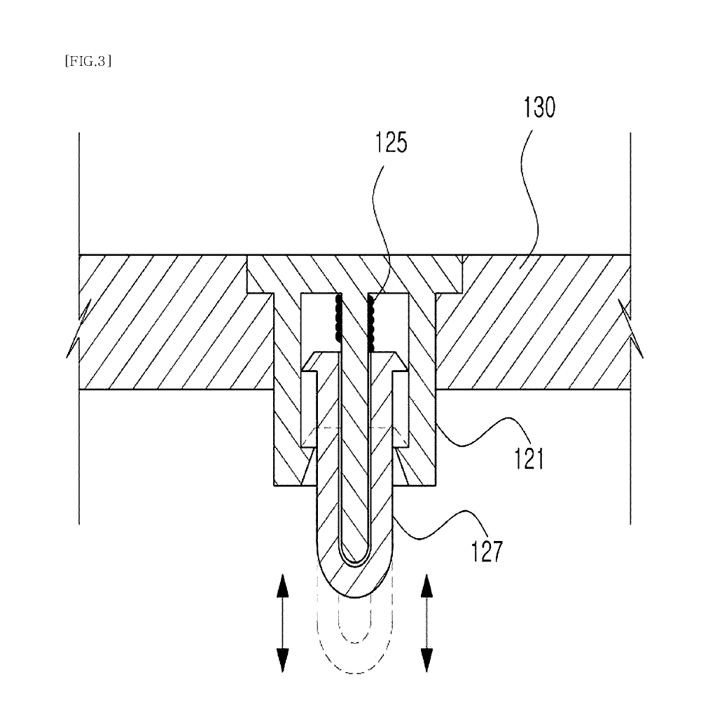

A human body impedance measurement device including a stress sensor will be described with reference to FIGS. 1 to 4. FIG. 1 is a top plan view illustrating a human body impedance measurement device including a stress sensor according to an exemplary embodiment, FIG. 2 is a cross-sectional view illustrating electrodes provided in the human body impedance measurement device according to the exemplary embodiment, and FIG. 3 is a cross-sectional view illustrating an operation state of the electrodes provided in the human body impedance measurement device according to the exemplary embodiment.

The human body impedance measurement device 10a according to the present exemplary embodiment is a case where a stress sensor is added to a substrate in the related art.

Particularly, a base plate 130a includes branch-leaf portions B1 radially extended from a center c thereof. In the present exemplary embodiment, a total of 12 branch-leaf portions B1 is provided. Electrodes 120 are arranged on a lower surface of each branch-leaf portion B1 and stress sensors 110 are arranged on a lower surface of each branch-leaf portion B1. The base plate 130a is formed of an easily bendable material, such as a flexible substrate.

The stress sensors 110 are arranged along the branch-leaf portions B1, and when the branch-leaf portion B1 is bent, each branch-leaf portion B1 is bent in accordance with a curve shape of a part of a human boy, which is in contact with the branch-leaf portion B1. In this case, the stress sensor 110 may detect intensity and a direction of stress according to the bending. Accordingly, when detection signals of the stress sensors 110 arranged along each of the branch-leaf portions B1 are combined, it is possible to calculate a 3D shape of the human body, to which the human body impedance measurement device 10a is applied.

An input electrode and an output electrode are connected to each power source. Further, the electrodes 120 may be arranged so as to be orthogonal in a circumferential direction and a radial direction. Further, a distance between the electrodes may be determined within a range of 5 mm to 20 mm for resolution of a final product according to a calculation of impedance.

The electrodes in the art may be used as the electrodes include in the human body impedance measurement device. As illustrated in FIGS. 2 and 3, the electrode 120 includes a housing member 121, a guide rod 123, a hollow electrode member 127, and an elastic member 125.

The guide rod 123 is extended to one opened surface of the housing member 121, and the hollow electrode member 127 may reciprocate in a state where the guide rod 123 is inserted into the hollow electrode member 127. In this case, uniform elastic force is applied to an external side of the hollow electrode member 127 by the elastic member 125.

The hollow electrode member 127 is formed of a conductive material or an electrode coated with a conductive material. The conductive material may include a material, for example, a gold electrode or a gold coated electrode, harmless to a human body.

The housing member 121 including the guide rod 125 is formed of a conductive material or an electrode coated with a conductive material. As long as a material has excellent conductivity, it is not necessary to particularly limit the material as the conductive material. However, like the hollow electrode member 127, a gold electrode or a gold coated electrode may also be used, and the housing member 121 may also be formed of a copper wire or an iron wire. An opened longitudinal outer circumference surface of the housing member 121 is formed to have a latching jaw 129 at an internal side thereof, thereby preventing the hollow electrode member 127 from being separated to the outside.

The elastic member 125 may also be formed of a conductive material, for example, a spring formed of metal.

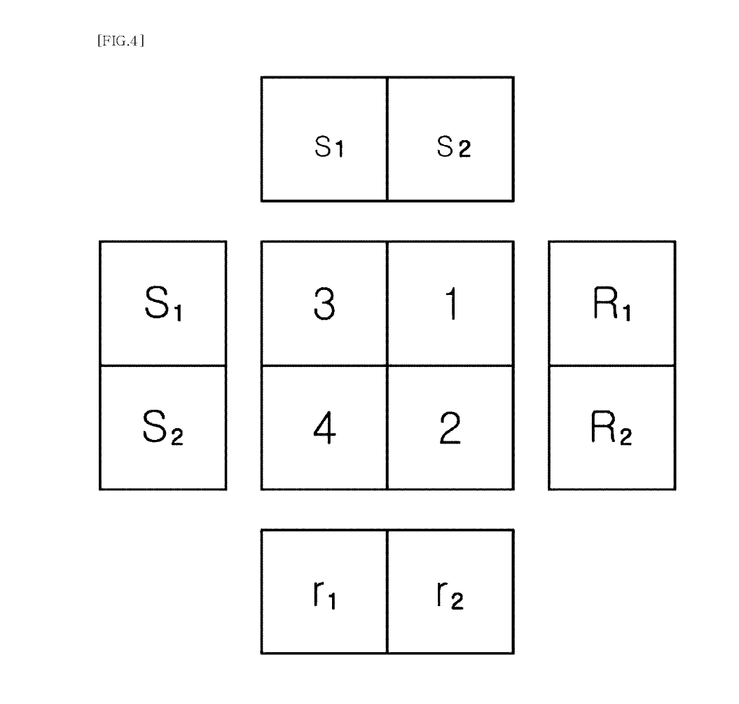

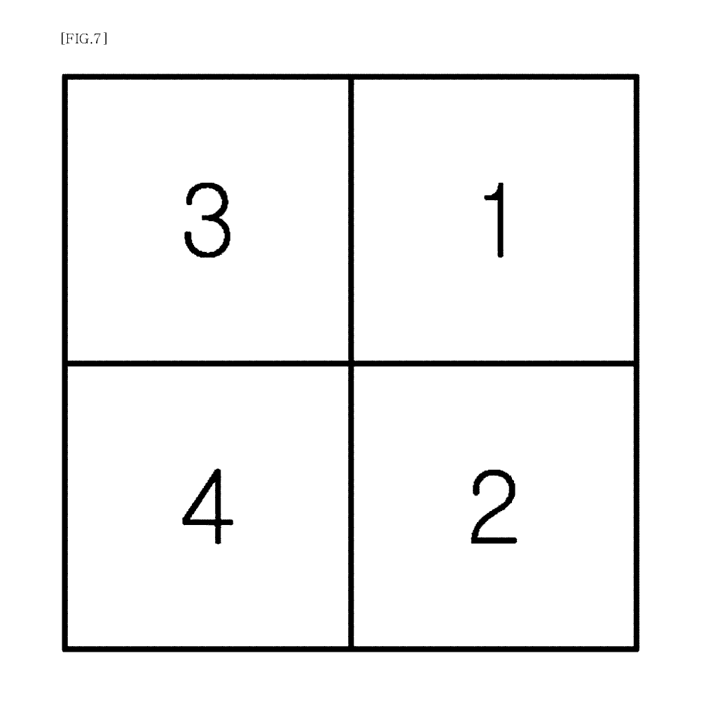

A process of imaging impedance inside a human body by using an impedance value measured by the EIT will be described with reference to FIGS. 4 to 7. FIGS. 4 to 7 are schematic diagrams for describing a process of imaging impedance inside a human body by using an impedance value measured by the EIT.

The EIT is a technology which is capable of showing an electric characteristic of a body section, and in the EIT, several electrodes are attached to body parts, a current sequentially flows in the electrodes, resistance is measured, and then the resistance inside the body is imaged. To this end, it is assumed that input electrodes S and s and receiving electrodes R and r are attached to a body tissue in a form of 2×2, and resistance is measured by making a current flow to the electrodes.

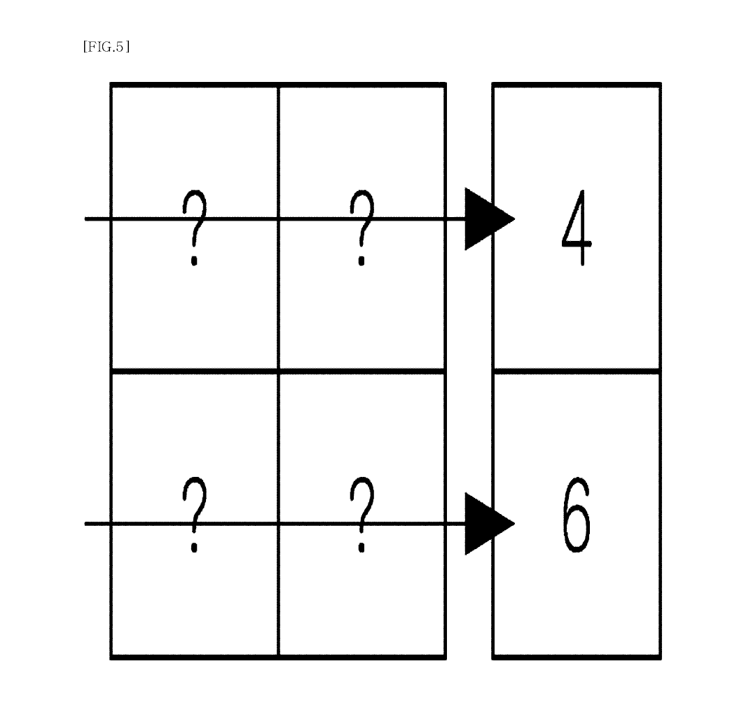

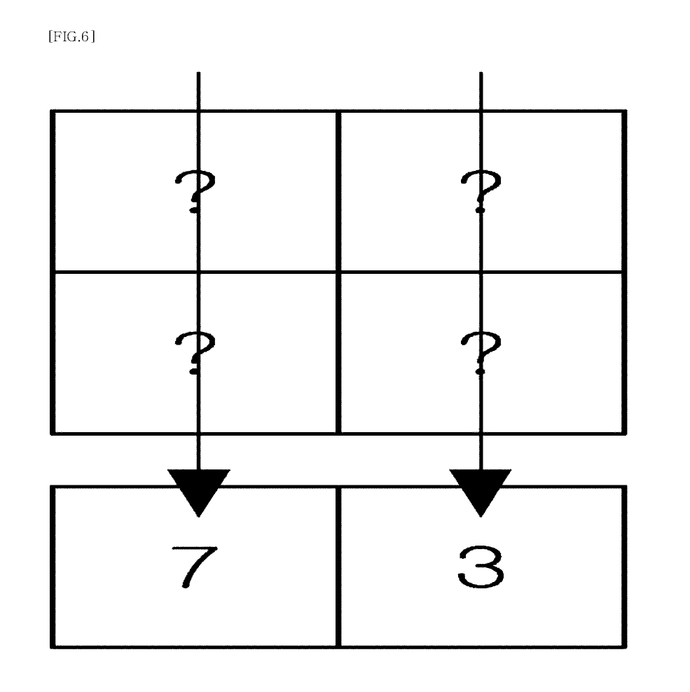

In this case, as illustrated in FIG. 4, horizontal input electrodes S₁ and S₂ and horizontal receiving electrodes R₁ and R₂, and vertical input electrodes s₁ and s₂ and vertical receiving electrodes r₁ and r₂. Next, as illustrated in FIG. 5, impedance in a horizontal direction is measured by making a current flow from the horizontal input electrodes S₁ and S₂ to the horizontal receiving electrodes R₁ and R₂. Next, as illustrated in FIG. 6, impedance in a vertical direction is measured by making a current flow from the vertical input electrodes s₁ and s₂ to the vertical receiving electrodes r₁ and r₂.

An inverse non-linear data process is performed by using the measured impedance values, it is possible to estimate a distribution of the impedance values in the corresponding body part.

An EIT apparatus is formed in a cylindrical ring shape, and is attached to a body in a form surrounding a whole body or attached to a wrist, an ankle, and the like, and then measures resistance by making a current sequentially flow. For example, the horizontally measured resistance and the vertically measured resistance correspond to a sum of total resistance of the body tissue, so that it is possible to detect a distribution of resistance values of the tissue transmitted to the section. As another method, after a distribution of resistance values is recognized, a voltage distribution of the human body is calculated according to intensity of a current, and an equipotential line position is displayed.

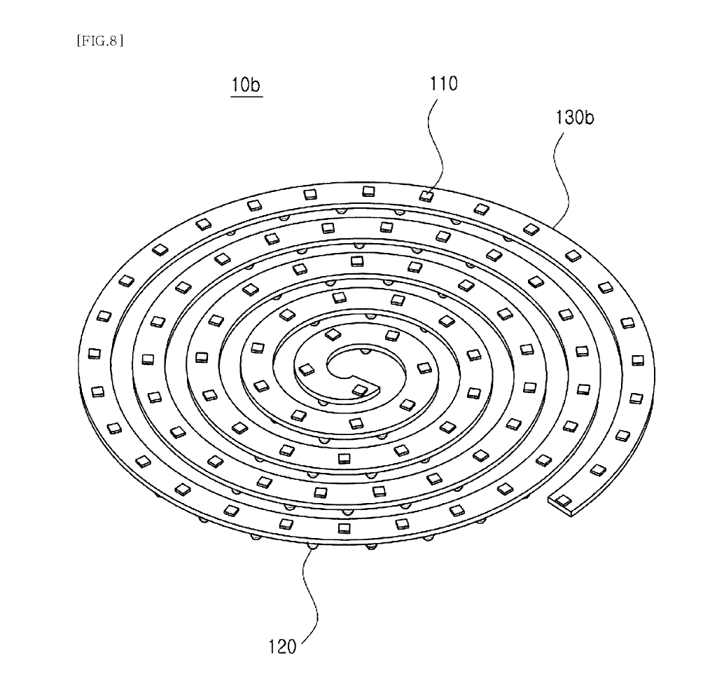

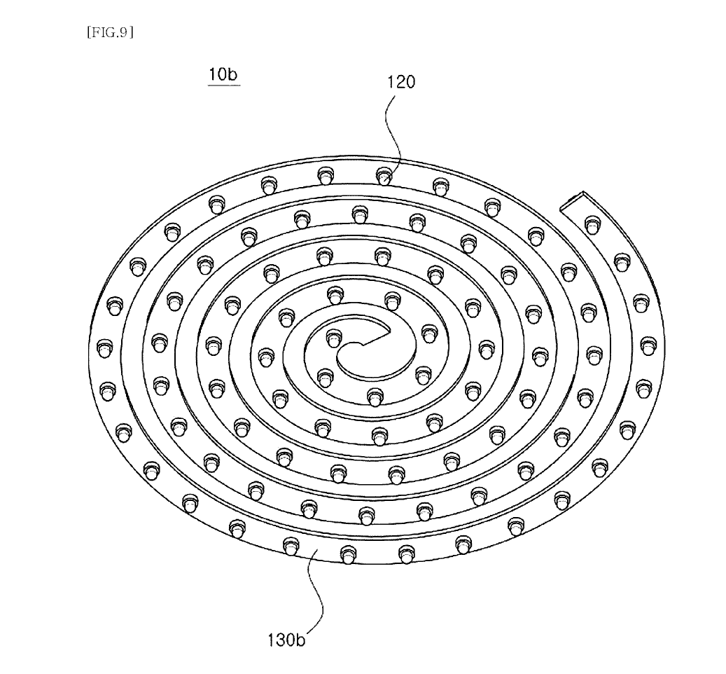

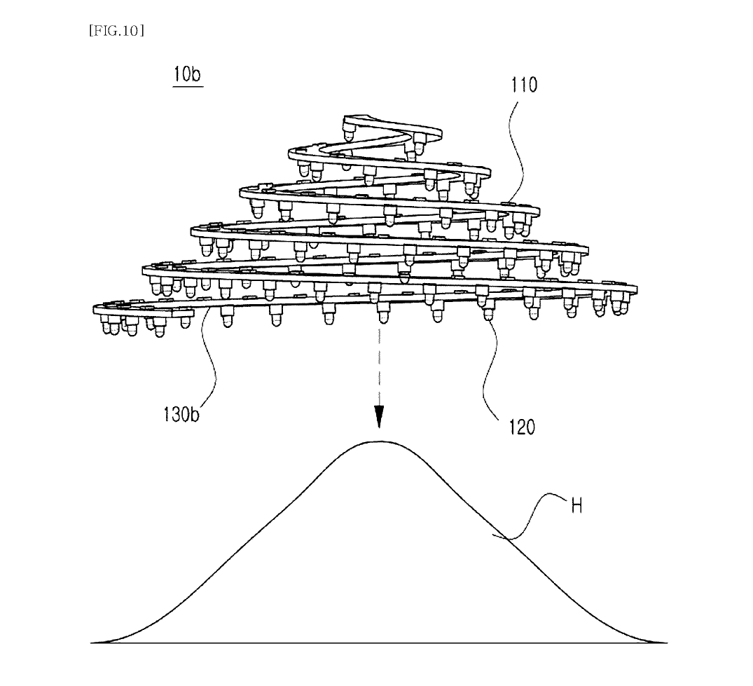

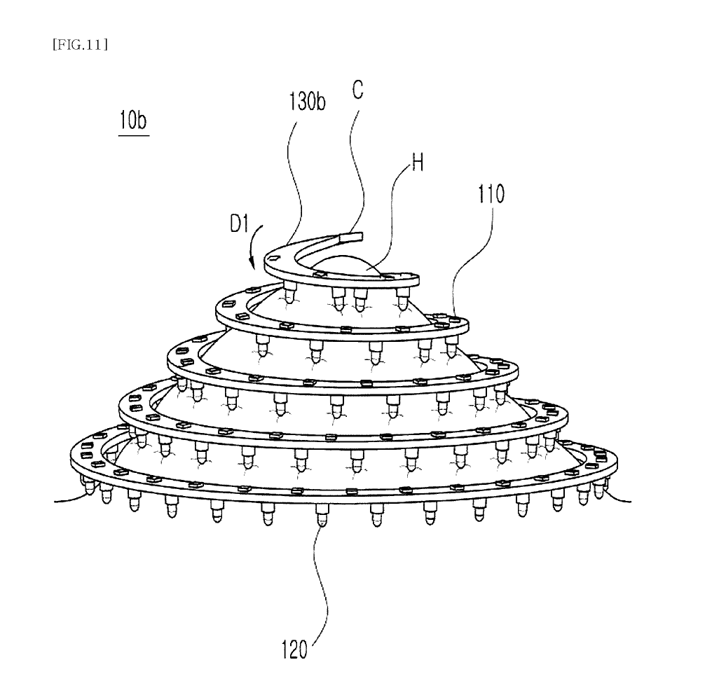

A spiral human body impedance measurement device will be described with reference to FIGS. 8 to 11. FIG. 8 is a perspective view illustrating a spiral human body impedance measurement device according to an exemplary embodiment of the present invention, FIG. 9 is a bottom perspective view illustrating the spiral human body impedance measurement device according to the exemplary embodiment of the present invention, FIGS. 10 and 11 are schematic diagrams illustrating an application of the spiral human body impedance measurement device according to the exemplary embodiment to a human body.

As illustrated in FIG. 8, a human body impedance measurement device 10b includes a spiral base plate 130b. That is, the base plate 130b is formed to have a length rotating in a spiral shape from a center thereof.

Stress sensors 110 are provided on the base plate 130b. In this case, the stress sensors 110 may be formed in a longitudinal direction of the base plate 130b with a predetermined interval according to a purpose, but the interval between the stress sensors 110 may be adjusted so that the stress sensors 110 are spirally arranged based on a center portion.

Referring to FIG. 9, a plurality of electrodes 120 is arranged on a lower surface of the base plate 130b. The electrodes 120 may also be formed in the longitudinal direction of the base plate 130b with a predetermined interval like the stress sensors, and the interval between the electrodes 120 may be adjusted so that the electrodes 120 are spirally arranged based on a center portion.

Further, each of the electrodes is connected to an input power line and an output power line like that of the aforementioned exemplary embodiment.

As illustrated in FIG. 10, in a case where the human body impedance measurement device 10b is applied to a part of a human body H having a protruding shape, the human body impedance measurement device 10b has a high and a low in accordance with an external curve surface of the human body H by a characteristic of the spiral base plate 130b.

As illustrated in FIG. 11, when the human body impedance measurement device 10b is in contact with the human body H, an external corner portion of the base plate 130b exhibits a bending characteristic according to the curve surface of the human body H by a curve of the human body H and gravity, and the degree of bending may be detected by the stress sensor 110 arranged at each part.

In the meantime, when external force is applied to the human body impedance measurement device 10b by using a separate cover and the like, the degree of bending according to the curve is further increased, so that the degree of bending is more easily detected by the stress sensor 110.

In the meantime, the human body impedance measurement device 10b may further include a shape calculating unit (not illustrated), which receives data about the degree of bending for each part of the base plate transmitted from the stress sensor, and calculates a 3D shape of the human body, to which the base plate is applied. It is difficult to estimate a shape of the human body, to which the base plate is applied, only with the impedance itself measured by the electrodes 120. Accordingly, a 3D shape including an accurate curve of the human body is estimated, and the estimated 3D shape is used for calculating impedance, thereby more accurately obtaining a result.

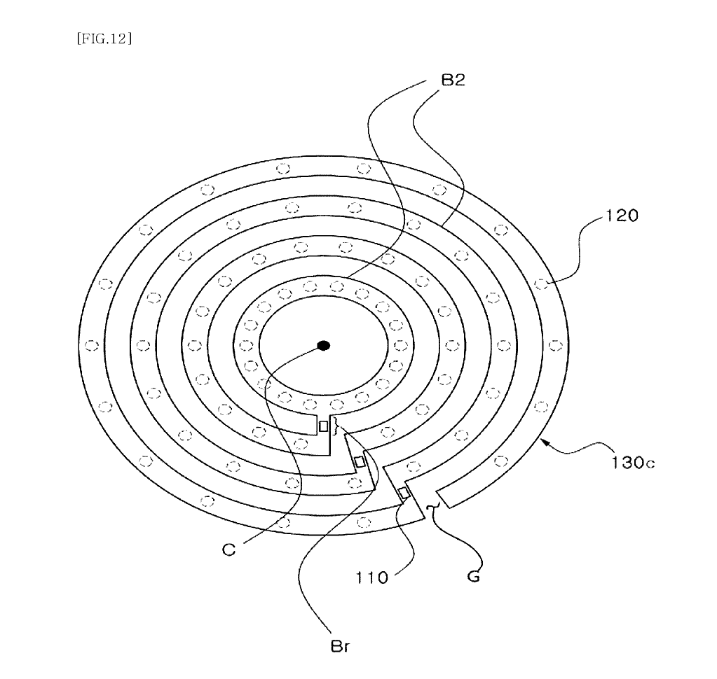

Another spiral human body impedance measurement device according to another exemplary embodiment will be described with reference to FIG. 12. FIG. 12 is a top plan view illustrating a spiral human body impedance measurement device according to another exemplary embodiment.

A base plate 130c according to the present exemplary embodiment includes a plurality of circular band bases B2, and base bridges Br connecting the circular band bases B2.

The circular band bases B2 are formed to have different diameters, and the circular band bases B2 are formed to maintain a predetermined interval. In this case, the circular band base B2 is formed with a gap portion G radially incised at a predetermined position. In this case, the base bridge Br connects an end portion of any one of the plurality of circular band bases B2 and an end portion of an adjacent outer circular band base B2.

The base plate 130c according to the present exemplary embodiment is formed to mostly have a predetermined radius by the shape of each circular band base B2, but the circular band bases B2 are connected by using the base bridges Br, so that a spiral connection structure is generally implemented. According to the structural characteristic, the base plate 130c according to the present exemplary embodiment has advantages in that the shape of the base plate 130c is freely transformed in accordance with a curve of the human body, and it is easy to arrange the electrodes to be orthogonal in a circumferential direction or in a radial direction.

In the meantime, in the structure, stress applied to the base plate 130c is mainly concentrated to the base bridge Br. In the present exemplary embodiment, in order to detect the stress, a stress sensor 110 may be provided on the base bridge Br.

In the meantime, additional stress sensors may be further provided.

In the meantime, an average interval of the gap portion G may be 5 mm to 20 mm in response to a distance between the electrodes to prevent an interval between the electrodes in the vicinity of the gap portion G between the electrodes from being larger than those of other parts.

In the above, the exemplary embodiments of the present invention have been described, but the technical spirit of the present invention is not limited to the aforementioned exemplary embodiments, and various modifications may be made within the scope of the technical spirit of the present invention embodied in the claims.

1. A device for measuring impedance within a human body, comprising:
a spiral base plate;
a plurality of electrodes arranged along the spiral base plate; and
a plurality of first and second power lines connected to the plurality of electrodes.

2. The device of claim 1, wherein a stress sensor, which detects a degree of bending of the spiral base plate, is provided at least a part of spaces between the electrodes arranged along the spiral base plate.

3. The device of claim 2, further comprising:
a shape calculating unit configured to calculate a 3D shape of the human body, to which the base plate is applied, from data about the degree of bending for each part of the base plate transmitted from the stress sensor.

4. The device of claim 1, wherein the plurality of electrodes is arranged from a center of the base plate in a radial direction.

5. The device of claim 1, wherein the base plate includes:
a plurality of circular band bases, which is formed to have a gradually increased radius and is spaced apart from one another by a predetermined interval, and has a gap portion having a radially incised shape at a part of the circular band base; and
a base bridge configured to connect an end portion of any one of the plurality of circular band bases and an end portion of an adjacent outer circular band base.

6. The device of claim 5, wherein an average interval of the gap portion is 5 mm to 20 mm.

7. The device of claim 6, wherein an interval of the gap portion is formed so as to correspond to a distance between the electrodes.

8. The device of claim 5, wherein a stress sensor, which detects a degree of bending of the base bridge, is provided on the base bridge.

9. The device of claim 1, wherein the distance between the electrodes is 5 mm to 20 mm.

10. The device of claim 1, wherein the first power line and the second power line are an input electrode and a receiving electrode, respectively.

11. A device for measuring impedance within a human body, comprising:
a flexible base plate;
a plurality of electrodes arranged in a first direction and a second direction orthogonal to the first direction on the flexible base plate;
a plurality of stress sensors arranged on the flexible base plate and configured to detect a degree of bending of the flexible base plate; and
a plurality of first and second power lines connected to the plurality of electrodes.

12. The device of claim 11, wherein the flexible base plate is formed with branch-leaf portions radially extended from a center portion, and
the electrode and the stress sensor are arranged on the branch-leaf portion.

13. The device of claim 11, wherein the flexible base plate is spirally formed, and
the electrode and the stress sensor are arranged along the spiral flexible base plate.

14. The device of claim 13, wherein the base plate includes:
a plurality of circular band bases, which is formed to have a gradually increased radius and is spaced apart from one another by a predetermined interval, and has a gap portion having a radially incised shape at a part of the circular band base; and
a base bridge configured to connect an end portion of any one of the plurality of circular band bases and an end portion of an adjacent outer circular band base.

15. The device of claim 14, wherein the stress sensor is provided on the base bridge and detects a degree of bending of the base bridge.

16. The device of claim 11, further comprising:
a shape calculating unit configured to calculate a 3D shape of the human body, to which the base plate is applied, from data about the degree of bending for each part of the base plate transmitted from the stress sensor.

17. The device of claim 13, wherein the plurality of electrodes is arranged from a center of the base plate in a radial direction.

18. The device of claim 11, wherein a distance between the electrodes is 5 mm to 20 mm.

19. The device of claim 11, wherein the first power line and the second power line are an input electrode and a receiving electrode, respectively.
